(19) United States
(12) Patent Application Publication
(10) Pub. No.: US 2025/0099677 A1
(43) Pub. Date: Mar. 27, 2025

(54) Method and Apparatus for Infusion Control

(71) Applicant: Medcaptain Medical Technology Co., Ltd., Shenzhen (CN)

(72) Inventors: Ping LI, Shenzhen (CN); Wenhui YANG, Shenzhen (CN); Haoke HUANG, Shenzhen (CN); Yazhou TANG, Shenzhen (CN)

(73) Assignee: Medcaptain Medical Technology Co., Ltd., Shenzhen (CN)

(21) Appl. No.: 18/885,713

(22) Filed: Sep. 15, 2024

(30) Foreign Application Priority Data
Sep. 22, 2023 (CN) 202311253090.0

(51) Int. Cl. A61M 5/168; A61M 5/142

(52) U.S. Cl. CPC A61M 5/1684; A61M 2005/14208; A61M 2205/3334

(57) ABSTRACT

A method for controlling infusion of a medication into a patient includes obtaining a rise time of a second infusion device; determining a first infusion end time of a first infusion device; and outputting a pre-start message to the second infusion device at a time at as far in advance of the least before the first infusion end time as the rise time. The second infusion device reaches a target infusion rate before the first infusion end time, so that after the end of the infusion of the first infusion device, the second infusion device can directly start the infusion at the target infusion rate. This avoids large flow rate fluctuations and provides continuous, uninterrupted infusion at a desired, more stable rate, reducing the probability of large changes in the patient's vital signs and improving the safety of the infusion.

### REFERENCE TO RELATED APPLICATIONS

The invention claims priority of Chinese Patent Application No. 202311253090.0, entitled “Infusion Control Method, Device and Storage Medium” filed with the China National Intellectual Property Administration on Sep. 22, 2023, which is incorporated herein by reference in its entirety.

### TECHNICAL FIELD

This invention generally relates to the field of medical technologies, and more particularly, to an infusion control method, device and corresponding computer-executable code stored in a storage medium.

### BACKGROUND ART

With the development of medical technology, infusion devices with higher accuracy and safety have been widely used. These typically use a mechanical driving force to accurately control the number of drops and flow rate of infusion to ensure that the medication can accurately and safely enter the patient's body.

However, the volume of medication that can be infused by the infusion device at any one time is always limited. In the treatment of critically ill patients, there is usually a need for continuous and long-term infusion, which inevitably leads to situations where medication or pumps need to be replaced. During the process of replacing medications or pumps, fluctuations in the infusion rate are inevitable. Significant fluctuations in the infusion rate can affect the patient's vital signs, and even endanger the patient's life in severe cases.

Therefore, there is a need to provide an infusion control method to reduce fluctuations in the infusion rate caused by medication or pump replacements, thereby improving infusion safety.

### SUMMARY OF THE INVENTION

The invention provides an infusion control method, a device, and a corresponding computer-executable code stored in a storage medium for solving the problem of how to reduce changes in the flow rate of a medication due to replacing medications, pumps, and the like, so as to improve the safety of the infusion.

In a first aspect, the invention provides an infusion control method, comprising:
obtaining a rise time of a second infusion device; wherein the rise time is the time required by the second infusion device to transition from initiating a second infusion operation to reaching a target infusion rate;
determining an infusion end time of the first infusion device; and
outputting a pre-start message/command to the second infusion device at least as far in advance of the infusion end time as the rise time.

In an embodiment, the rise time is related to the target infusion rate.

In an embodiment, the step of determining the infusion end time of the first infusion device comprises:
obtaining an infusion state of the first infusion device, and determining the infusion end time of the first infusion device based on the infusion state.

In an embodiment, the method further comprises:
displaying infusion information in a display screen; wherein the infusion information comprises one or more of: an infusion trend graph, infusion sequence information, infusion state information, the infusion end time, and the rise time.

In an embodiment, the method further comprises:
sending infusion parameter information of the first infusion device to the second infusion device.

In an embodiment, after the step of outputting the pre-start message to the second infusion device, the method further comprises:
issuing an up-to-standard message when an actual infusion rate of the second infusion device reaches the target infusion rate;
outputting a failure alarm upon detecting that the first infusion device has not received the message from the second infusion device within a preset time after the infusion end time of the first infusion device.

In an embodiment, the method further comprises:
switching the second infusion device to being the device to perform the infusion when the second infusion device reaches the target infusion rate and the first infusion device ends its infusion operation.

In a second aspect, the invention provides an infusion control method applied to a second infusion device in an infusion system, the infusion system comprising a first infusion device and the second infusion device, the method comprising:
obtaining a target infusion rate;
determining a rise time to reach the target infusion rate based on the target infusion rate and a predetermined calculation rule; and
outputting the rise time to the first infusion device.

In an embodiment, the rise time is further related to at least one of a drive parameter of the second infusion device and a specification parameter of a syringe connected to the second infusion device.

In an embodiment, the method further comprises:
performing a pre-start based on a received pre-start message; and
outputting an up-to-standard message when an actual infusion rate reaches the target infusion rate.

In an embodiment, the method further comprises:
displaying infusion information in a display screen; wherein the infusion information comprises one or more of: an infusion trend graph, infusion sequence information, infusion state information, an infusion end time, and the rise time.

In a third aspect, the invention provides an electronic medical device, comprising: a processor, and a memory communicatively connected to the processor;
wherein the memory is configured to store computer-executable instructions; and the processor is configured to execute the computer-executable instructions stored in the memory to implement any one of the above-mentioned methods.

In a fourth aspect, the invention provides a computer-readable storage medium, wherein the computer-readable storage medium stores computer-executable instructions, and the computer-executable instructions are configured to implement any one of the above-mentioned methods.

In a fifth aspect, the invention provides a computer program product comprising a computer program, wherein the computer program when executed by a processor is configured to implement any one of the above-mentioned methods.

The invention provides an infusion control method, device, and corresponding computer-executable code stored in a storage medium. The method includes: obtaining a rise time of a second infusion device; wherein the rise time is the time required by the second infusion device from initiating an infusion to reaching a target infusion rate; determining an infusion end time of the first infusion device; and outputting a pre-start message to the second infusion device at least as far in advance of the infusion end time as the rise time In the infusion control method of the invention, the pre-start message is sent to the second infusion device at least as far in advance of the infusion end time as the rise time, so as to instruct the second infusion device to reach the target infusion rate before the end of the infusion of the first infusion device, so that the second infusion device can start the infusion at the target infusion rate directly after the end of the infusion of the first infusion device, thereby avoiding large infusion rate fluctuations in the process of convergence of the infusion device. Therefore, continuous infusion can be carried out at a desired and more stable infusion rate, reducing the probability of large changes in the patient's vital signs, and improving the safety of the infusion.

### BRIEF DESCRIPTION OF THE DRAWINGS

The accompanying drawings herein, which are incorporated into and form a part of the specification, illustrate embodiments consistent with the invention and are used in conjunction with the specification to explain the principles of this invention.

FIG. 1 shows a flowchart of an infusion control method provided by an embodiment of the invention.

FIG. 2a shows a schematic diagram of a steady-state infusion setting interface provided by an embodiment of the invention.

FIG. 2b shows a schematic diagram of an infusion parameter setting interface provided by an embodiment of the invention.

FIG. 3 shows a schematic flowchart of yet another infusion control method provided by an embodiment of the invention.

FIG. 4 shows a schematic flowchart of still another infusion control method provided by an embodiment of the invention.

FIG. 5 shows an infusion trend diagram provided by an embodiment of the invention.

FIG. 6 shows a schematic diagram of an infusion interface applied to a first infusion device provided by an embodiment of the invention.

FIG. 7 shows a schematic diagram of an infusion interface applied to a second infusion device provided by an embodiment of the invention.

FIG. 8 shows a schematic diagram of yet another infusion interface applied to the second infusion device provided by an embodiment of the invention.

FIG. 9 shows a schematic diagram of still another infusion interface applied to the second infusion device provided by an embodiment of the invention.

FIG. 10 is a schematic diagram of an infusion interface provided by an embodiment of the invention after the display information is changed.

FIG. 11 is a schematic structural diagram of an infusion control device provided by an embodiment of the invention.

FIG. 12 is a schematic structural diagram of another infusion control device provided by an embodiment of the invention.

FIG. 13 is a schematic structural diagram of an electronic medical device provided by an embodiment of the invention.

By means of the above accompanying drawings, definite embodiments of the invention have been shown, which will be described in greater detail later. These accompanying drawings and textual descriptions are not intended to limit the scope of the invention idea by any means, but rather to illustrate the concepts of the invention for those skilled in the art by reference to particular embodiments.

### DETAILED DESCRIPTION

Example embodiments will be described herein in detail and are illustrated in the accompanying drawings. When the following description relates to the accompanying drawings, the same numerals in the different accompanying drawings indicate the same or similar elements unless otherwise indicated. The embodiments described in the following example embodiments do not represent all embodiments consistent with the invention. Rather, they are only examples of devices and methods consistent with some aspects of this invention as detailed in the appended claims.

The terms “first”, “second”, “third”, “fourth” and the like, if any, in the specification and claims of the present application and the accompanying drawings described above are used to distinguish similar objects and need not be used to describe a particular order or sequence. It should be understood that the data so used may be interchangeable, where appropriate, so that the embodiments of the present invention described herein can, for example, be practiced in an order other than those illustrated or described herein. In addition, the terms “include” and “have”, and any variations thereof, are intended to cover non-exclusive embodiments, e.g., processes, methods, systems, products or devices that include a series of steps or units need not be limited to those clearly listed, but may include other steps or units that are not clearly listed or that are inherent to those processes, methods, products or devices.

Critically ill patients require the use of vasoactive agents to improve blood pressure, cardiac output, and microcirculation due to the presence of hemodynamic instability during treatment. Vasoactive agents are medications that are used to combat shock by modulating vasodilatation, altering vascular function, and improving microcirculatory perfusion, and are commonly used, such as dopamine, sodium nitroprusside, and epinephrine. Some intensive care patients often need to use vasoactive medications continuously. However, in the process of changing medications and pumps, there are bound to be flow rate fluctuations, which will affect the patient's vital functions and even endanger their lives. How to minimize flow rate changes due to medication and pump changes to ensure that the flow rate is stable during the continuous use of vasoactive medications in patients and thus improve infusion safety is a current problem to be solved.

In order to solve the above problem, the invention provides an infusion control method, in which the rise time required for the second infusion device to reach a target infusion rate from initiating the infusion is pre-calculated, and a pre-start message is sent to the second infusion device at least as far in advance of the end of the first infusion as the rise time, so as to enable the second infusion device to reach the target infusion rate, which is preferably the same as or close to the ongoing infusion rate of the first infusion device, before the end of the infusion of the first infusion device. The second infusion device thus infuses the patient at the target infusion rate after the end of infusion of the first infusion device. In this way, the infusion flow rate does not change too much when changing pumps or medications, and the flow rate remains stable, which can reduce the safety hazard caused by the change of flow rate due to the change of medications or pumps, and thus improve the safety of the infusion.

The technical solution of the invention and how it solves the above technical problems are described in detail below with specific embodiments. The following specific embodiments may be combined with each other, and the same or similar concepts or processes may not be repeated in certain embodiments. Embodiments of the invention will be described below in conjunction with the accompanying drawings.

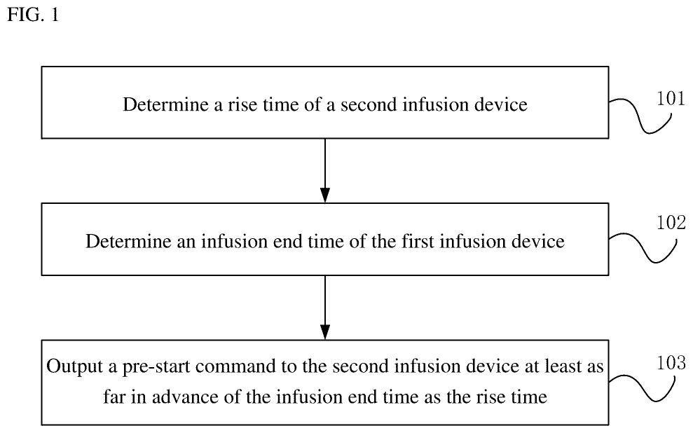

FIG. 1 shows a flowchart of an infusion control method provided by an embodiment of the invention. The system that implements embodiments of the invention may be an infusion control device, which may be disposed on at least one electronic medical device in an infusion system. The electronic medical device may be, for example, a first infusion device, an infusion central station, an infusion workstation, an infusion monitoring system, a mobile terminal or a computer used in conjunction with the medical device, which is not limited in this invention.

In addition, the infusion system also includes a second infusion device. The first infusion device and the second infusion device involved in the invention may be an infusion pump, a syringe pump, or the like, for controlling the infusion process parameters such as the flow rate of a medicament. The first infusion device and the second infusion device are connected by wired and/or wireless means to transmit information to each other so as to realize steady-state infusion based on the method of the invention.

For example, the first infusion device and the second infusion device may be connected wired through network cable, coaxial cable, fiber optics, etc., for inter-transmission of data. A communication link between the first infusion device and the second infusion device may also be established via wireless technology such as WiFi, 4G, 5G, Bluetooth, and Zigbee, to provide a basis for data communication between the first infusion device and the second infusion device. A combination of one or more of these may be selected to realize the connection in practical applications to better realize the steady-state infusion. In one embodiment, the first infusion device and the second infusion device may also be plugged into an infusion workstation to realize a wired connection.

When there are n (n≥2) infusion devices involved for continuous steady-state infusion, the infusion control device of the embodiment may also be disposed on the preceding (n−1) infusion devices, thereby realizing continuous steady-state infusion of multiple infusion devices.

As an example, the embodiment of the invention is illustrated with the implementing system being the infusion control device. As shown in FIG. 1, the embodiment provides an infusion control method including the following steps.
S101, a rise time of the second infusion device is obtained. The rise time is a time required by the second infusion device to transition from initiating a second infusion operation to reaching a target infusion rate.

As an example, steady-state infusion may involve a plurality of infusion devices for continuous infusion, including at least a first infusion device and a second infusion device. The first infusion device refers generically to an infusion device that is infusing or is about to start infusion, and the second infusion device refers generically to the next infusion device after the first infusion device. The purpose of the steady-state infusion is to control the flow rate of the medicament solution to be stable during the entire infusion process. This avoids too great a fluctuation in the flow rate, which may affect vital functions of the patient or even endanger the life of the patient. In the invention, the infusion control device needs to first obtain a rise time of the second infusion device. The rise time is the time required for the second infusion device to reach the target infusion rate from initiating infusion. It should be noted that the “initiating infusion” herein is not the actual initiation of infusing the patient, but is only a pre-initiation of the infusion operation, so that the infusion device reaches the target infusion rate before infusing the patient. The rise time is related to one or more of a drive parameter of the second infusion device, a specification parameter of a syringe connected to the second infusion device, and a target infusion rate.

As an example, the drive parameter of the second infusion device may be an intrinsic parameter related to its ability to drive the flow of the medicament. This may, for example, be related to a driving distance parameter of the infusion device, such as a transmission gap or a push head gap, but also related to a driving force parameter of the infusion device, such as a driving force, a driving power, and a stiffness of the infusion device. Because of machining errors, the drive parameters of each infusion device will have some differences, which can be obtained through testing and calibration before leaving the factory. The specification parameter of the syringe may include parameters such as the diameter, length, and capacity of the syringe, or may be a comprehensive parameter obtained by determining the above-mentioned parameters.

In one example, the rise time may be obtained according to the following calculation formula:

$${t}_{0} = \frac{M}{ν * A} * K \qquad ( 1 )$$

t₀ is the rise time, M may be the drive parameter of the second infusion device in mm, v may be a target infusion rate of the second infusion device in ml/h, A is a parameter determined for the specific syringe in mm/h, which may be obtained by dividing the length of the syringe by the capacity. K is a calibration coefficient which may be determined by per-device testing or other experimental means and which is negatively correlated with the speed of the moving pusher (piston/plunger/etc.) head up the infusion device. The smaller the speed, the larger K. The calibration coefficient K is generally greater than or equal to 1, which is not limited in this invention. Accordingly, the unit of the calculated rise time t₀ is h, which may be converted to other units according to practical needs, for example, to minutes and the like.

Therein, the rise time of the second infusion device may be calculated by any medical device in the infusion delivery system, and then shared to other medical devices that need to use the data, which is not limited in the invention. In practice, the rise time may be obtained by the device for calculating the rise time after obtaining each parameter in the formula (1) and bringing it into the formula.

As an example, the respective parameters of the above formula may be obtained by evaluating different parameter settings for the respective infusion device while it performs steady-state infusion prior to actual infusion of a patient. Setting the parameters may be done by the user inputting them directly, or the corresponding values of some parameters may be pre-set, and the corresponding parameter values may be applied after being selected by the user. For example, parameters such as a medication name, a medication concentration, a target infusion rate, a flow rate, and a preset volume may be input/selected, and may also be selected and applied after information such as the brand, model, and specification of the infusion device is input/selected to obtain the corresponding drive parameters of the second infusion device, which is not limited in the invention.

In some examples, some or all of the infusion parameters of the plurality of infusion devices performing the steady-state infusion are the same. In order to save time for parameter setting and also to improve the user experience, the infusion control method of the invention may further include sending the infusion parameter information of the first infusion device to the second infusion device.

As an example, before actual infusion, the plurality of infusion devices for performing steady-state infusion and their respective infusion parameters may be set, such as the medication name, the medication concentration, the target infusion rate, the flow rate, and the preset volume. If same-medication infusion is used or the infusion parameters of each infusion device are the same, the infusion parameters of each infusion device may be designed to be able to be synchronized with one key, and the parameters that have been set up can be synchronized to other infusion devices. For example, if the infusion parameter information of the first infusion device is the same as the infusion parameter information of the second infusion device, after the infusion parameter information of the first infusion device has been set, the infusion parameter information of the first infusion device can be sent directly to the second infusion device to save time in setting the parameters.

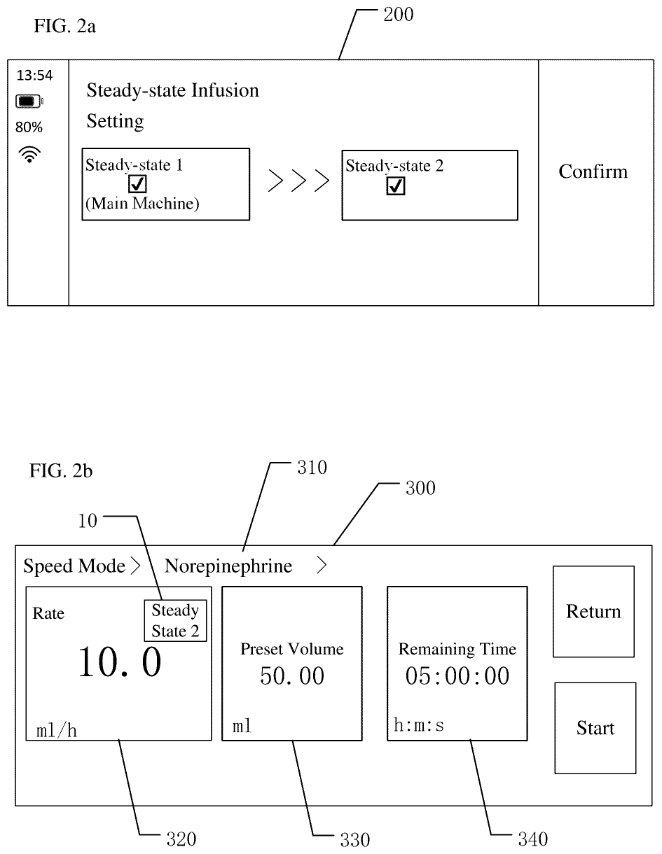

As an example, FIG. 2a shows a schematic diagram of a steady-state infusion setting interface provided by an embodiment of the invention. As shown in FIG. 2a, using the steady-state infusion setting interface 200, each infusion device may be set to carry out steady-state infusion, and the infusion sequence information of each infusion device can be determined. In this figure, the illustrated interface includes a steady state 1 and a steady state 2, and the infusion device of the steady state 2 will continue to perform the infusion after the infusion of the device of the steady state 1 (the local machine) is finished.

As an example, FIG. 2b shows a schematic diagram of an infusion parameter setting interface provided by an embodiment of the invention. As shown in FIG. 2b, using the infusion parameter setting interface 300, parameters such as the infusion medication 310, the target infusion rate 320, the preset volume 330, and the remaining time 340 may be set for each infusion device. The “steady state 2” icon 10 indicates setting of the infusion parameters of the infusion device for the steady state 2. In this figure, the infusion medication is norepinephrine, the target infusion rate is 10.0 ml/h, the preset volume is 50.00 ml, and the remaining time is 5 h.

FIG. 2a and FIG. 2b illustrate one of many examples, and other parameters may be displayed in the setting interface of the practical application, which are not limited in the invention.
S102 (FIG. 1): an infusion end time of the first infusion device is determined.

As an example, in order to realize steady-state infusion using the method of embodiments of this invention, the infusion control device also signals the second infusion device to prepare to be at its infusion rate before the end of the infusion of the first infusion device. Thus, the infusion end time of the first infusion device also needs to be determined.

As an example, the step of determining the infusion end time of the first infusion device may include obtaining an infusion state of the first infusion device, and determining the infusion end time of the first infusion device based on the infusion state.

As an example, the infusion state of the first infusion device may be monitored, and based on the specifics of the infusion state, information such as the actual remaining volume of infusion, and the actual infusion rate of the first infusion device may be obtained, and then the infusion end time may be determined based on such information.
S103, a pre-start message is output to the second infusion device at least as far in advance of the of the infusion end time as the rise time.

As an example, after determining the rise time of the second infusion device and the infusion end time of the first infusion device, the infusion control device outputs the pre-start message to the second infusion device at least as far in advance of the infusion end time as the rise time. The pre-start message is configured to instruct the second infusion device to pre-start so that the second infusion device reaches a target infusion rate before the first infusion device ends the infusion and the second infusion device can take over supplying medication to the patient upon switch-over from the first infusion device.

For example, if it is determined that the rise time required for the second infusion device to reach the target infusion rate from initiating the infusion is 10 min, the pre-start message should be sent to the second infusion device at least 10 min prior to the end of the infusion of the first infusion device, so that the second infusion device can increase its output rate to the target infusion rate. Following this, after the end of the infusion of the first infusion device, the second infusion device may directly start the infusion at the target infusion rate. This thus avoids large flow rate fluctuations in the process of convergence of the infusion devices in order to provide continuous uninterrupted infusion at a desired, more stable infusion rate in continuous infusion, reducing the probability of large changes in the patient's vital functions and improving the safety of the infusion.

As an example, if the infusion control device is used to command the first infusion device, in the step of outputting the pre-start message to the second infusion device, the pre-start message may be sent directly from the first infusion device to the second infusion device, or the pre-start message may be sent by the first infusion device to the infusion workstation/infusion central station first, and then be forwarded by the infusion workstation/infusion central station to the second infusion device. If the infusion control device is configured to cooperate with the infusion workstation/infusion central station, the infusion workstation/infusion central station may send the pre-start message to the second infusion device after determining the rise time of the second infusion device and the infusion end time of the first infusion device, and before the rise time from the infusion end time of the first infusion device.

Regardless of the manner in which the pre-start message is sent as described above, it is only necessary for the second infusion device to receive the pre-start message long enough before the end of the infusion of the first infusion device for it to get its output rate up to the target infusion rate in advance.

The infusion control method provided embodiments of the invention, include: obtaining a rise time of the second infusion device, in which the rise time is a time required by the second infusion device from initiating infusion to reaching the target infusion rate; determining an infusion end time of the first infusion device; and outputting the pre-starting message to the second infusion device at least as far in advance of the end time of the infusion as the rise time. The infusion control device sends the pre-start message to the second infusion device at least as far in advance of the end time of the infusion of the first infusion device as the rise time to instruct the second infusion device to increase its output rate to reach the target infusion rate before the end time of the infusion of the first infusion device, so that the second infusion device can start the infusion at the target infusion rate directly after the infusion end time of the first infusion device. This thus avoids large flow rate fluctuations in the process of convergence of the infusion devices in order to provide continuous, uninterrupted infusion at a desired, more stable infusion rate thereby reducing the probability of large changes in the patient's vital signs and improving the safety of the infusion.

In some examples, if the infusion control device used together with an infusion workstation/infusion central station, and the infusion control is performed by the infusion workstation/infusion central station, after outputting the pre-start message to the second infusion device, the infusion control method of the invention may further include the following steps:

An up-to-standard message is issued when the actual infusion rate of the second infusion device reaches a target infusion rate. A failure alarm is output when the central station detects that the first infusion device has not received the up-to-standard message from the second infusion device within a preset time before an end of the infusion of the first infusion device.

As an example, the infusion workstation/infusion central station and the like may also be configured to monitor the infusion states of the first infusion device and the second infusion device, and to issue the up-to-standard message when the actual infusion rate of the second infusion device reaches the target infusion rate, so as to prompt the first infusion device or the relevant person that the actual infusion rate of the second infusion device has reached the target infusion rate, and that the switching of equipment can be carried out. In addition, the workstation/infusion central station may determine whether the first infusion device receives the up-to-standard message from the second infusion device within a preset time before the end of the infusion. If the central station confirms that the first infusion device received the up-to-standard message of the second infusion device within the preset time before the end of the infusion, the normal operation of the respective devices is continued, or an explicit prompt characterizing that the up-to-standard message of the second infusion device has been received is issued. And if it is determined that the first infusion device has not received the up-to-standard message of the second infusion device within the preset time before the end of the infusion, the failure alarm is output to prompt the user to carry out abnormality processing as soon as possible.

The invention does presuppose or require any specific value of the preset time. For example, the preset time may be set to a value greater than or equal to 0 according to practical demand. When the preset time is 0, that is, when the first infusion device is just at the end of the infusion, the failure alarm may be issued when the up-to-standard message from the second infusion device is not yet received at the end of the infusion of the first infusion device, so as to allow more time for pre-starting for the second infusion device, so that it reaches the target flow rate of the infusion as closely as possible before the start of the second device's infusion. More preferably, to allow time for the user to deal with an abnormal situation, the preset time is set to 30 s or 1 min or some other smaller value greater than 0, which can allow time for the user to carry out processing of the fault without causing too great an impact on the infusion result.

In addition, self-monitoring may be performed by the first infusion device, and if the up-to-standard message from the second infusion device is not received within the preset time before the end of the infusion, the failure alarm is output.

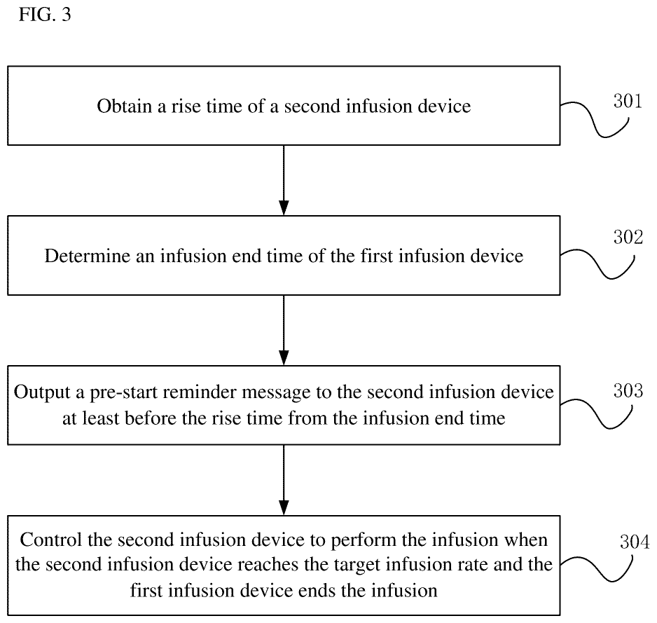

FIG. 3 shows a flowchart of yet another infusion control method according to an embodiment of the invention. As shown in FIG. 3, if the infusion control device may be connected to communicate with an infusion workstation/infusion central station, such that infusion control is performed by the infusion workstation/infusion central station. The infusion control method of the invention may include the following steps.
S301, a rise time of the second infusion device is obtained. The rise time is the time required for the second infusion device to transition from initiating an infusion to reaching a target infusion rate.
S302, an infusion end time of the first infusion device is determined.
S303, a pre-start message is output to the second infusion device at least until the rise time from the infusion end time.

The specific implementation of steps S301-S303 is similar to steps S101-S103, and will therefore not be repeated here.
S304, when the second infusion device reaches the target infusion rate and the first infusion device stops infusing, the second infusion device is switched to being the device that performs the infusion.

As an example, if the first infusion device and the second infusion device are continuously operating, the infusion workstation/infusion central station may also control the second infusion device to continue the infusion at the target infusion rate when it is determined that the second infusion device has reached the target infusion rate and the first infusion device has finished its phase of the infusion.

The target infusion rate of the first infusion device and the target infusion rate of the second infusion device may be the same or different. If they are the same, the entire infusion process may be realized to be infused at a stable flow rate, thereby improving the safety of the infusion. If they are different, the difference in the flow rate fluctuation caused by switching the infusion device may also be reduced, and a relatively stable output process may also be realized, thereby improving the safety of the infusion.

The infusion control method provided by an embodiment of the invention includes: obtaining a rise time of the second infusion device, in which the rise time is the time required for the second infusion device to reach a target infusion rate from when it initiates infusion; determining an infusion end time of the first infusion device; outputting a pre-start message to the second infusion device at least before the rise time from the infusion end time; and controlling the second infusion device to perform the infusion when the second infusion device reaches the target infusion rate and the first infusion device ends the infusion. The infusion control method of the invention can automatically control the second infusion device to start the infusion at the target infusion rate after the first infusion device has finished its phase of the infusion. This avoids large flow rate fluctuations in the process of convergence of the infusion devices in order to provide continuous, uninterrupted infusion at a desired, more stable infusion rate, reducing the probability of large changes in the patient's vital signs and improving the safety of the infusion.

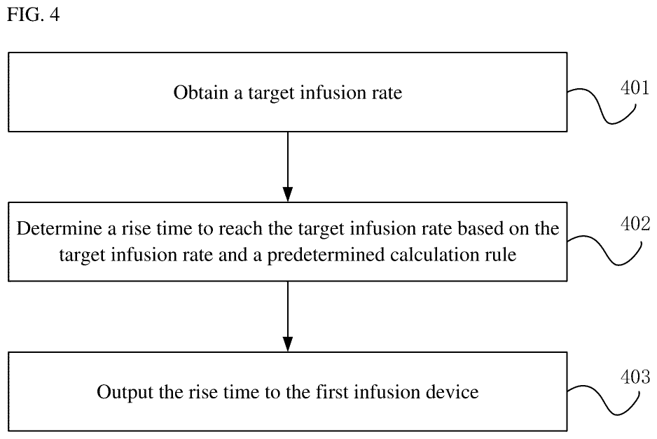

FIG. 4 shows a flowchart of still another infusion control method provided by an embodiment of the invention. The infusion control method provided by this embodiment is applied to a second infusion device in an infusion system. The infusion system includes a first infusion device and a second infusion device.

When there are n (n≥2) infusion devices involved in the continuous steady-state infusion, the infusion control method provided in this embodiment may also be applied to other subsequent infusion devices other than the first infusion device, so as to realize continuous steady-state infusion provided by multiple infusion devices.

As shown in FIG. 4, the infusion control method provided by the embodiment comprises:
S401, a target infusion rate is obtained.

As an example, the second infusion device first obtains its own target infusion rate, which may be obtained by the user when performing parameter setting. If it is the same as the target infusion rate of the first infusion device, it may also be acquired by the first infusion device when performing information synchronization, although this is not required by the invention.
S402: a rise time to reach the target infusion rate is determined based on the target infusion rate and a predetermined calculation rule.

As an example, the second infusion device may determine the rise time for reaching the target infusion rate based on the target infusion rate and the predetermined calculation rule after acquiring its target infusion rate.

The rise time is a function of one or more of: a drive parameter of the second infusion device, a specification parameter of a syringe connected to the second infusion device, and the target infusion rate.

As an example, the drive parameter of the second infusion device, which may be an intrinsic parameter of the infusion device, to drive the flow of the medicament, may be related not only to a driving distance parameter of the infusion device, such as a transmission gap, a push head gap, but also to a driving force parameter of the infusion device, such as a driving force, driving power, and a stiffness of the infusion device. Because of machining errors, the drive parameters of each infusion device will have some differences, which can be determined through testing and calibration before leaving the factory. The specification parameter of the syringe may include parameters such as the diameter, length, and capacity of the syringe, or may be a comprehensive parameter obtained by determining the above-mentioned parameters.

As an example, the predetermined calculation rule may be chosen according to predetermined calculation formula, and the predetermined calculation formula may be formula (1) above, the specific realization of which has been described in the foregoing embodiments and will not be repeated herein.
S403, the rise time is output to the first infusion device.

As an example, when the second infusion device determines the rise time, the rise time is also output to the first infusion device. Given the rise time sent by the second infusion device, the first infusion device can determine the length of time required by the second infusion device from initiating its phase of infusion to reaching the target infusion rate, thereby determining when to send the pre-start message to the second infusion device.

The first infusion device and the second infusion device may be connected by wired and/or wireless means. For example, the second infusion device may directly output the rise time to the first infusion device, or may first send the rise time to the infusion workstation/infusion central station, which then forwards it to the first infusion device.

In some examples, the infusion control method provided by the embodiment, further includes performing a pre-start based on a received pre-start message.

As an example, the pre-start message may be issued prior to the rise time from an infusion end time of the first infusion device, and the second infusion device may perform a pre-start procedure after receiving the pre-start message, to enable the second infusion device to reach the target infusion rate before the end of infusion of the first infusion device.

In some examples, the infusion control method provided in this embodiment, further includes outputting an up-to-standard message when an actual infusion rate reaches the target infusion rate.

As an example, when the actual infusion rate reaches the target infusion rate, the second infusion device may also output the up-to-standard message to prompt the first infusion device or a relevant person that the actual infusion rate of the second infusion device has reached the target infusion rate, and that device switching can be performed.

The embodiment provides an infusion control method applied to a second infusion device in an infusion system, in which the infusion system includes a first infusion device and the second infusion device. The method includes: obtaining a target infusion rate; determining a rise time to reach the target infusion rate based on the target infusion rate and a predetermined calculation rule; and outputting the rise time to the first infusion device. As a result, the first infusion device may determine the length of time required by the second infusion device from initiating the infusion to reaching the target infusion rate based on the rise time sent by the second infusion device, thereby determining the time for sending the pre-initiation message to the second infusion device, so as to ensure that the second infusion device may reach the target infusion rate before the end of the infusion of the first infusion device. Accordingly, after the end of infusion by the first infusion device, when infusion is switched to the second infusion device, the second infusion device is able to directly start infusing at the target infusion rate. This avoids large flow rate fluctuations in the process of convergence of the infusion devices in order to provide continuous, uninterrupted infusion at a desired, more stable infusion rate, reducing the probability of large changes in the patient's vital functions and improving the safety of the infusion.

In addition, in various electronic medical devices such as the first infusion device, the second infusion device, the infusion workstation, the infusion central station, and the like involved in the invention for performing the steady-state infusion control, each device may also include a display screen, and the infusion control method of the invention may also include displaying infusion information in a display screen; wherein the infusion information comprises one or more of: an infusion trend graph, an infusion sequence information, an infusion state information, an infusion end time, and the rise time.

As an example, the infusion trend graph may depict a trend of change in the actual infusion rate with the infusion time; infusion sequence information may indicates an infusion sequence of each of the infusion devices; and infusion status information may indicate an infusion state of each of the infusion devices.

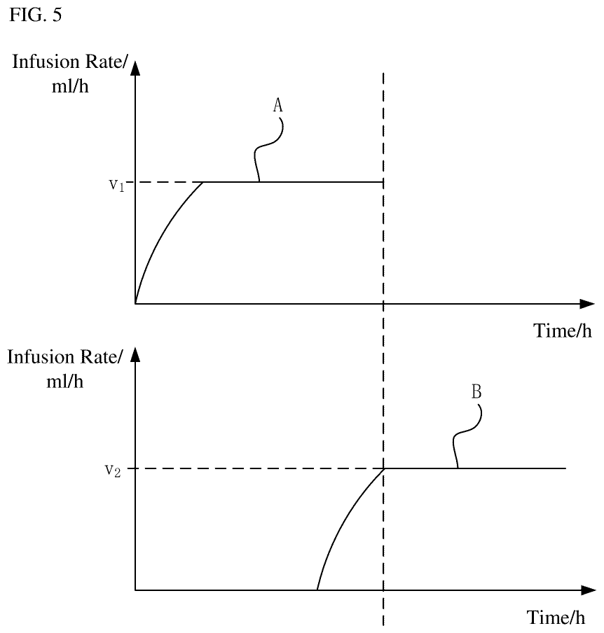

As an example, FIG. 5 shows an infusion trend graph for an embodiment of the invention. As shown in FIG. 5, the infusion trend line A is a trend of the infusion rate of the first infusion device as a function of infusion time, and the infusion trend line B is a trend of the infusion rate of the second infusion device as a function of infusion time. The horizontal axis is the infusion time, and the vertical axis is the infusion rate. After the infusion rate of the first infusion device rises from 0 to the target infusion rate v1, infusion is carried out at v1 until the end of the infusion first phase of the infusion. The second infusion device starts pre-starting before the end of the first infusion phase. When the first infusion device stops infusing, the infusion rate of the second infusion device will already have reached the target infusion rate v2. Then, after switching to infusion by the second infusion device, the second infusion device may directly carry out infusion at v2.

In addition, the infusion trend line A and the infusion trend line B may be shown as using the same coordinate system, as shown in FIG. 5, or using different coordinate systems. Alternatively, v1 and v2 may be equal or different.

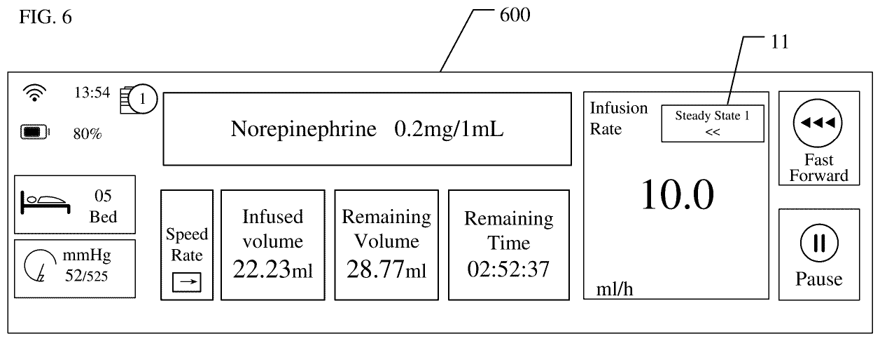

As another example, FIG. 6 shows a schematic diagram of an infusion interface for the first infusion device according to an embodiment of the invention. As shown in FIG. 6, in the infusion interface 600, the icon 11 indicates “Steady State 1”, indicating that the interface of the display shows the infusion information of the first infusion device in a steady-state infusion state. In the speed mode, in the illustrated example, the medication being infused by the first infusion device is norepinephrine, which has an already infused volume of 22.23 ml, a remaining volume of 28.77 ml, a remaining time of 02:52:37, and an infusion rate of 10.0 ml/h.

In addition, basic information such as network connection information, current time information, remaining power information, patient bed number, and infusion end time may be displayed in the interface 600. The infusion end time may be determined by the current time information as well as the remaining time of the infusion. Controls such as fast-forward and pause may optionally be provided in the interface 600 for the user to carry out the control. When the “Steady State 1” icon 11 is not displayed or is displayed as “none”, this may indicate that there is no other device that needs to continue to perform the infusion after the first infusion device has finished the infusion.

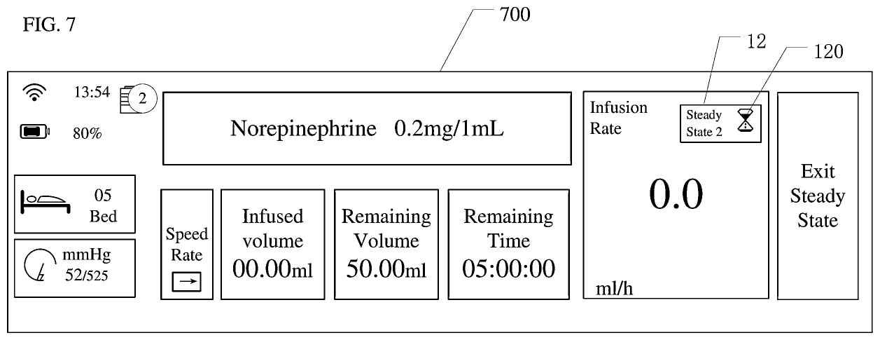

As an example, FIG. 7 shows a schematic diagram of an infusion interface for a second infusion device according to an embodiment of the invention. As shown in FIG. 7, in the infusion interface 700, the icon 12 is shown as “Steady State 2” to indicate that the displayed interface is showing the infusion information of the second infusion device. According to the display state of the illustrated hourglass icon 120, it can be determined that it is a pre-start state or an infusion waiting state or an infusing state. FIG. 7 shows a schematic diagram of an infusion interface of the second infusion device when it enters the pre-start state. At the moment of entering the pre-start state, the infused volume is 0.00 ml and the remaining volume is the same as the preset volume, which is still 50.00 ml. The remaining time is still 05:00:00, and the infusion rate is also 0.0 ml/h. Upon entering the pre-start state, the illustrated hourglass icon 120 may become vertically dynamic.

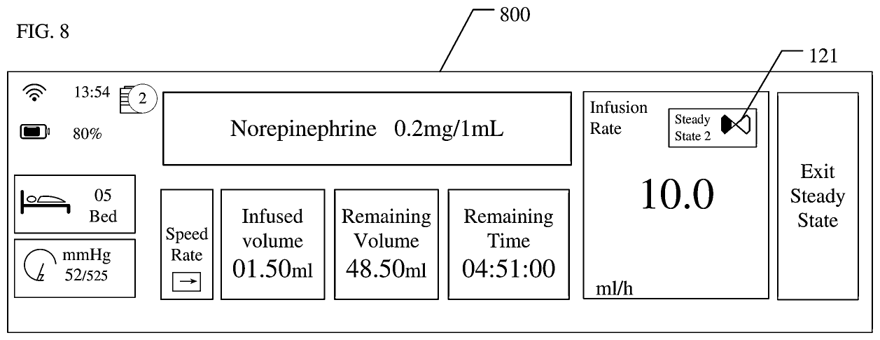

As an example, FIG. 8 shows a schematic diagram of yet another infusion interface that may be used for the second infusion device provided by an embodiment of the invention. On the basis of the interface in the pre-start state shown in FIG. 7, when the pre-start is finished, i.e., after the infusion rate reaches the target infusion rate, the interface may switch to the infusion interface 800 as shown in FIG. 8. The second infusion device is initially in the infusion waiting state, and the illustrated hourglass icon 121 is horizontally static during the waiting process.

After finishing the pre-start procedure and entering the waiting state, however, the volume already output by the second device may have been, for example, 01.50 ml, the remaining volume may be 48.50 ml, the remaining time becomes 04:51:00 and the infusion rate becomes the target infusion rate of 10.0 ml/h. Based on the volume of the change in the remaining time, the rise time can be determined to be 10 min, which may also be displayed in the interface; this is not a limitation of the invention, however.

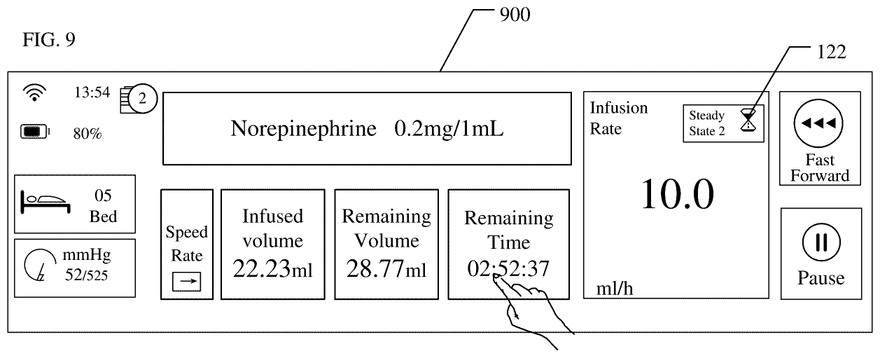

As an example, FIG. 9 shows a schematic diagram of still another infusion interface that may be generated for the second infusion device provided by an embodiment of the invention. Starting from the interface of the infusion waiting state shown in FIG. 8, when the first infusion device has finished the infusion, display may be switched to the second infusion device to continue the infusion at the target infusion rate, and the interface changes to the infusion interface 900 as shown in FIG. 9. At this time, the second infusion device is in the state of the actually infusing the patient, and the illustrative hourglass icon 122 is vertically dynamic to indicate that infusion is in progress. When the infusion state is entered, the infused volume, the remaining volume, and the remaining time change as the infusion proceeds may be displayed, In this figure, the infused volume is shown as being 22.23 ml, the remaining volume is shown as 28.77 ml, the remaining time is shown as being 02:52:37, and the infusion rate stabilizes at a target infusion rate of 10.0 ml/h.

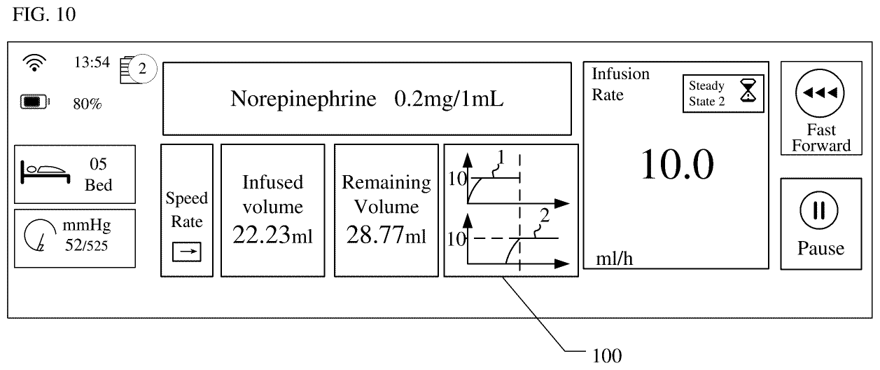

In some examples, an infusion trend graph may also be displayed in the display interface. As an example, FIG. 10 shows a schematic diagram of an infusion interface provided by an embodiment of the invention after the display information is changed. If the user clicks the remaining time in the display interface as shown in FIG. 9, the display content of the region may be changed to the infusion trend graph 100 as shown in FIG. 10. In addition, the user may click on other regions in the graph may optionally to display other information,

The examples depicted in FIGS. 5-10 above are only intended to be examples of what can be realized by the invention, and the specific values of each parameter in the graphs are only examples and should not be regarded as limitations of the invention. In addition to the information already shown in the figures, more information may be displayed according to the practical needs, such as parameter setting, personnel, and healthcare personnel duty information, in order to fully display all the key information and facilitate the ability the healthcare personnel to view and obtain the data.

The following are examples of device embodiments of the invention that can be used to perform the method embodiments of the invention. For details not disclosed in the device embodiments of the invention, please refer to the method embodiments of the invention.

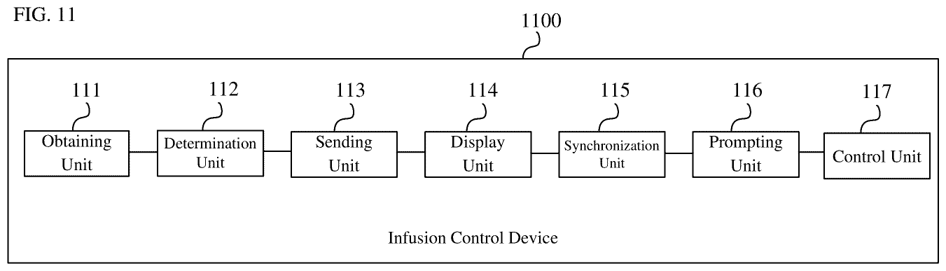

FIG. 11 shows a schematic structural diagram of an infusion control device provided by an embodiment of the invention. As shown in FIG. 11, the infusion control device 1100 provided by the embodiment of the invention includes an obtaining unit 111, a determination unit 112, and a sending unit 113.

The obtaining unit 111 is configured to obtain the rise time of the second infusion device. As described above, the rise time is the time required by the second infusion device from initiating an infusion to reaching a target infusion rate.

The determination unit 112 is configured to determine an infusion end time of the first infusion device.

The sending unit 113 is configured to output a pre-start message to the second infusion device at no later before the infusion end time than the rise time.

In one embodiment, the rise time is related to a target infusion rate.

In one embodiment, the determination unit 112, is specifically configured to obtain an infusion state of the first infusion device and determine an infusion end time of the first infusion device based on the infusion state.

In one embodiment, the device 1100 further includes a display unit 114.

The display unit 114 is configured to display infusion information in a display screen. The infusion information includes one or more of an infusion trend graph, an infusion sequence information, an infusion state information, the infusion end time, and the rise time.

In one embodiment, the device 1100 further includes a synchronization unit 115, which is configured to send the infusion parameter information of the first infusion device to the second infusion device.

In one embodiment, the device 1100 further includes a prompting unit 116.

The prompting unit 116 is configured to send an up-to-standard message when the actual infusion rate of the second infusion device reaches a target infusion rate; and output a failure alarm when it is determined that the up-to-standard message of the second infusion device is not received within a preset time before the end of the infusion of the first infusion device.

In one embodiment, the device 1100 further includes a control unit 117.

The control unit 117 is configured to control the second infusion device to perform the infusion when the second infusion device reaches the target infusion rate and the first infusion device ends the infusion.

The device provided in this embodiment may be used to perform the method of the above embodiments, the realization principles and technical effects of which are similar and will not be repeated herein.

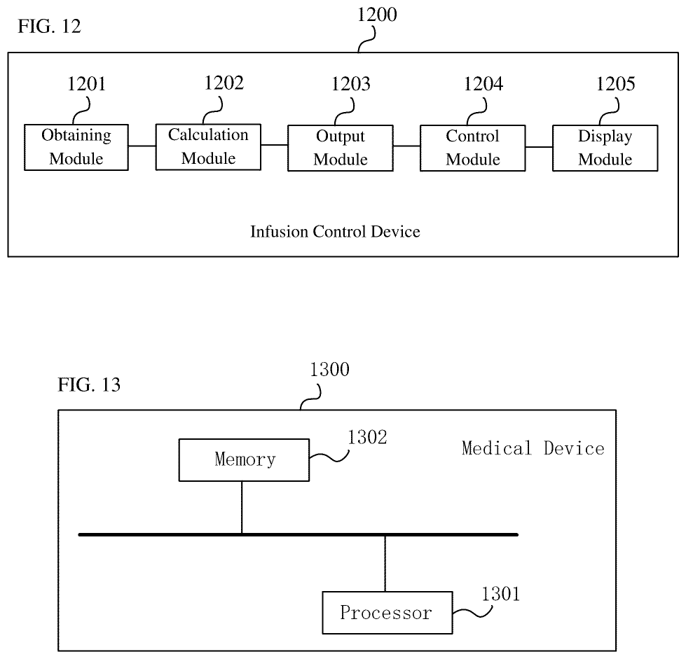

FIG. 12 shows a schematic structural diagram of an infusion control device provided by an embodiment of the invention. The infusion control device 1200 provided in the embodiments of the invention is applied to a second infusion device in an infusion system, the infusion system including a first infusion device and a second infusion device. As shown in FIG. 12, the infusion control device 1200 provided by an embodiment of the invention includes an obtaining module 1201, a calculation module 1202, and an output module 1203.

The obtaining module 1201 is configured to obtain a target infusion rate.

The calculation module 1202 is configured to determine a rise time for reaching the target infusion rate based on the target infusion rate and a predetermined calculation rule.

The output module 1203 is configured to output the rise time to the first infusion device.

In one embodiment, the rise time is also related to a drive parameter of the second infusion device and/or a specification parameter of a syringe connected to the second infusion device.

In one embodiment, the device 1200 further includes a control module 1204.

The control module 1204 is configured to perform a pre-start action based on the received pre-start message; and/or, output an up-to-standard message when the actual infusion rate reaches the target infusion rate.

In one embodiment, the device 1200 further includes a display module 1205.

The display module 1205 is configured to display infusion information in a display screen. The infusion information includes one or more of an infusion trend graph, an infusion sequence information, an infusion state information, an infusion end time, and a rise time.

The embodiment provides a device that can be used to perform the method of the above embodiment, the realization principle and technical effect of which are similar and will not be repeated herein.

It is to be noted that it should be understood that the division of the various modules of the above device is merely a logical, functional division, and may be fully or partially integrated into a physical entity or physically separated when actually realized. Moreover, these modules may all be realized in the form of software invoked through processing elements, or may also be realized in the form of hardware, or may also be part of the module through the processing elements invoked in the form of software, part of the module through the form of hardware. In addition, it may also be stored in the form of program code in the memory of the above device, which is called by one of the processing elements of the above device and performs the functions of the above data processing module. Other modules are realized similarly. In addition, all or some of these modules may be integrated together or may be realized independently. The processing element here may be an integrated circuit with signal processing capability. In the realization process, the steps of the above method or each of the above modules may be accomplished by integrated logic circuits of hardware in the processor element or by instructions in the form of software.

FIG. 13 shows a schematic structural diagram of an electronic medical device provided by an embodiment of the invention. The electronic medical device 1300, as shown in FIG. 13, includes: a processor 1301, and a memory 1302 communicatively connected to the processor.

The memory 1302 is configured to store computer-executable instructions, and the processor 1301 is configured to execute the computer-executable instructions stored in the memory 1302 to implement any one of the above-mentioned methods.

In the specific implementation of the above-described electronic device, it should be understood that the processor may be a Central Processing Unit (CPU), and may also be other general-purpose processor, a Digital Signal Processor (DSP), an Application Specific Integrated Circuit (ASIC), and so on. The general purpose processor may be a microprocessor or the processor may be any conventional processor. The method disclosed in conjunction with embodiments of the invention may be directly embodied in a hardware processor to perform the completion, or performed with a combination of hardware and software modules in the processor to perform the completion.

Embodiments of the invention also provide a computer-readable storage medium. The computer-readable storage medium has stored computer-executable instructions, which are configured to realize any one of the above-mentioned methods when executed by a processor.

It will be appreciated by those of ordinary skill in the art that all or some of the steps for implementing each of the above-mentioned method embodiments may be accomplished by hardware associated with the computer instructions. The above-mentioned program may be stored in a computer readable storage medium. The program, when executed, performs the steps including each of the above-described method embodiments. The above-mentioned storage medium includes: a ROM, a RAM, a magnetic disk, or a CD-ROM, and various other media that can store program code.

Embodiments of the invention also provide a computer program product including a computer program, which is configured to implement any one of the above-mentioned method when executed by a processor.

Other embodiments of this invention will readily come to mind to those skilled in the art upon consideration of the specification and practice of the invention disclosed herein. The invention is intended to cover any variations, uses, or adaptations of the invention that follow the general principles of the invention and include common knowledge or customary technical means in the art not disclosed herein. The specification and embodiments are to be regarded as examples only, and the true scope and spirit of this invention are indicated by the following claims.

It should be understood that this invention is not limited to the precise structure which has been described above and illustrated in the accompanying drawings, and that various modifications and changes may be made without departing from its scope. The scope of this invention is limited only by the appended claims.

1. A method for controlling infusion of a medication into a patient, comprising:
obtaining a rise time of a second infusion device, said rise time being a time required for the second infusion device to transition from initiating a second infusion operation to reaching a target infusion rate;
determining an end time of a first infusion by a first infusion device; and
transmitting a pre-start message to the second infusion device and thereby causing the second infusion device to initiate the second infusion operation at least as long in advance of the infusion end time as the rise time.

2. The method of claim 1, further comprising determining the rise time as a function of the target infusion rate.

3. The method of claim 1, wherein the step of determining the infusion end time of the first infusion of the first infusion device comprises:
obtaining an infusion state of the first infusion device, and determining the infusion end time of the first infusion of the first infusion device as a function of the infusion state.

4. The method of claim 1, further comprising:
displaying infusion information in a display screen; wherein the infusion information comprises at least one of: an infusion trend graph, an infusion sequence information, an infusion state information, the end time of the first infusion, and the rise time.

5. The method of claim 1, further comprising:
sending infusion parameter information of the first infusion device to the second infusion device and adjusting operational parameters of the second infusion device according to the infusion parameter information.

6. The method of claim 1, further comprising, after the step of transmitting the pre-start message to the second infusion device:
issuing an up-to-standard message when an actual infusion rate of the second infusion device reaches the target infusion rate;
outputting a failure alarm upon detecting that the first infusion device has failed to receive the message from the second infusion device within a preset time before an end of the infusion of the first infusion device.

7. The method of claim 1, further comprising:
controlling the second infusion device to perform the second infusion when the second infusion device reaches the target infusion rate and the first infusion device ends the infusion.

8. A method for controlling infusion by an infusion system of a medication into a patient, in which the infusion system includes a first infusion device and a second infusion device, the method comprising:
determining a target infusion rate corresponding to a first infusion rate of the first infusion device;
determining a rise time of the second infusion system to reach the target infusion rate as a function of the target infusion rate and a predetermined calculation rule; and
outputting the rise time to the first infusion device, said first infusion device thereupon transmitting a pre-start message to the second infusion device causing the second infusion device to initiate a second infusion operation at least as long in advance of a first infusion end time of the first infusion device as the rise time;
initiating the second infusion operation no later than at a time corresponding to the rise time before the first infusion end time;
switching infusion from the first infusion device to the second infusion device at the first infusion end time, whereby:
the second infusion device begins infusing the patient at the target infusion rate; and
the rate of infusion of the patient is maintained substantially constant when infusion is switched from the first to the second infusion device.

9. The method of claim 8, wherein the rise time is a function of at least one of: a drive parameter of the second infusion device and a specification parameter of a syringe connected to the second infusion device.

10. The method of claim 8, further comprising the second infusion device initiating the second infusion operation upon receipt of a pre-initiation message.

11. The method of claim 8, further comprising:
outputting an up-to-standard message when an actual infusion rate reaches the target infusion rate.

12. The method of claim 8, further comprising:
displaying infusion information on a display screen; wherein the infusion information comprises at least one of: an infusion trend graph, infusion sequence information, infusion state information, the first infusion end time, and the rise time.
